(19) United States
(12) Patent Application Publication
(10) Pub. No.: US 2021/0398648 A1
(43) Pub. Date: Dec. 23, 2021

(54) Method And Apparatus For Treatment And Relapse Prevention In Alcohol, Chemical, And Other Dependencies

(71) Applicant: Thomas Stevens, Moraga, CA (US)

(72) Inventor: Thomas Stevens, Moraga, CA (US)

(21) Appl. No.: 16/945,803

(22) Filed: Aug. 1, 2020

Related U.S. Application Data
(60) Provisional application No. 63/041,112, filed on Jun. 18, 2020.

(51) Int. Cl. G16H 20/70; G16H 50/70; G16H 10/60; G16H 50/30; G16H 40/67; G06Q 50/26; G16H 10/20; G06N 5/02; A61B 5/00; G09B 19/00

(52) U.S. Cl. CPC G16H 20/70; G16H 50/70; G16H 10/60; G16H 50/30; G16H 40/67; H04B 1/385; G16H 10/20; G06N 5/02; A61B 5/4845; G09B 19/00; G06Q 50/265

(57) ABSTRACT

An application on a user's mobile device provides interaction and network support which may be based or supplemented with knowledge of the user's emotional state and risk of drug or alcohol dependency relapse. The user's emotional state may be determined in a number of interrelated and/or independent ways including asking the user to rate his/her state, games/quizzes, and/or pictures of the user, any one or more of which may be analyzed for emotional cues. The application establishes an emotional baseline, identifies triggers, tracks movements/activities, and helps the user stay on the road to recovery through consistent messaging, advice, ideas, and feedback. The user's network is notified if conditions point to a likely or imminent relapse and a response team may be automatically selected and notified to intercede in a virtual and/or physical intervention.

### CROSS REFERENCE TO RELATED APPLICATIONS

This application claims priority to U.S. Provisional Patent Application Ser. No. 63/041,112, filed on 18 Jun. 2020, entitled “Method And Apparatus For Treatment And Relapse Prevention in Drug, Alcohol, and other Dependencies” which is hereby incorporated by reference in its entirety.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

### Field of Invention

The present invention relates to alcohol and chemical dependency related treatments, relapse prevention, ongoing recovery support.

### Description of Related Art

Various drug, alcohol, and other dependency treatments are known.

### SUMMARY OF THE INVENTION

The present inventor has realized the need for real-time evaluation of environment and circumstances for reminders, structured advice, warnings, and/or all-out intervention in dependency situations to facilitate treatment and to catch, stall, and respond before or within early stages of a relapse event. The present invention comes in many embodiments and no single feature or component of one embodiment is exclusive thereto or required in any other embodiment even if described or implied as important to a particular embodiment.

In one embodiment, an application is provided that runs on a individual/patient's mobile device, tracking movement and monitoring activities which are applied to statistical data collected from any or all of the patient, the patient's peer group, patient's friends/family/social network (including on-line presences, posts, contacts, etc.), and national data/statistics of other patients similarly situated—for example. Decisions may be made based on, for example, the statistical averages, Artificial Intelligence (AI) (e.g., decision rules vetted over a larger database of drug and alcohol based dependencies), and such vetting performed using a machine learning API or other source.

The events and activities monitored include emotional cues and events/activities related to emotional cues such that, for example, allow for an accurate assessment of an emotional state or current emotional stability of the individual. Such assessment may be compared with statistical averages or compared against the individual's personal history and/or correlated against other events that may also be tracked.

In one embodiment, various decisions or processes may be run on the user's mobile device, on a back-end process hosted in the cloud, and/or split between local processing and cloud based servers or other computing means. The data may be analyzed to produce alerts/notifications as, for example, set-up in the application wherein the user may, for example, grant permission (e.g., opt-in permissions for monitoring, scanning, review, voice recognition, voice-to-text, key-word search, mobile device control such as camera and microphone, location data, and other data/analysis, applied statistics, analytics, etc., some or all of which may otherwise be private or confidential). The notification may be for example, notification or reminders to the user and may include pop-up photos or other information, suggested courses of action, etc. Such notification and/or reminders may be designed, for example, to prevent, forestall (or stall enough time for an intervention) actions by the user that have been determined to be statistically likely such as the purchase of alcohol or tobacco.

Data collection may include standard data collection devices commonly included in a mobile device e.g., location data, inertial data (e.g. IMU/accelerometer data collection), etc., and/or specialized medical or data collection equipment such as wearables, patches, printed, implantable devices, etc. The data collected includes all types of biometric or physical data related to the user such as blood pressure/heart rate (e.g., full EKG), galvanic skin response, glucose, alcohol air analysis.

The alcohol air analysis may be provided, for example, by a breathalyzer like device that does not need to be breathed into—detecting the smallest quantities possible of alcohol or alcohol laced vapors—not necessarily from the user (e.g., could be the surrounding environment—such as a nearby restaurant serving alcohol). A positive analysis would likely raise the possibility of user consumption and may signal an alert to a team leader or case manager, for example.

In one embodiment, the medical equipment may comprise, for example, a commercially available fitness device. For example, an API or other interface configured to access a Fitbit or similar device. Such API may be configured to directly access the device, or access a cloud storage/database populated using data from the device, for example. Regardless of the methodology, the data may be collected for analysis and correlation to events, diary entries, doctor/therapist notations, etc., and may be used to find patterns of activity as they relate to the patient and/or his/her treatment.

The various embodiments may include or be realized as, for example, a device, method, or apparatus configured to track events and emotional status of an individual and correlate the events and emotional status against past events and emotional statuses to identify when overall circumstances for the individual have converged or are converging toward a likelihood of relapse. Notifying at least one of the individual, healthcare provider, therapist, or other stakeholders in the individual's recovery (friends, family, etc.) when such circumstances exist. Feedback to the user may be used, for example, to forestall the individual's behavior or change their course of action. Feedback to the individual may be used to help the individual recognize/acknowledge that current circumstances are not favorable and potentially allow them to recognize the danger and take some action to prevent full relapse.

Portions of the embodiments, whether a device, method, or other form, may be conveniently implemented in programming on a general purpose computer, or networked computers, and the results may be displayed on an output device connected to any of the general purpose, networked computers, or transmitted to a remote device for output or display. In addition, any components of any embodiment represented in one or more computer program or module(s), data sequence(s), and/or control signal(s) may be embodied as an electronic signal broadcast (or transmitted) at any frequency in any medium including, but not limited to, wireless broadcasts, and transmissions over copper wire(s), fiber optic cable(s), and co-ax cable(s), etc.

### BRIEF DESCRIPTION OF THE DRAWINGS

A more complete appreciation of the various embodiments and many of the attendant advantages thereof will be readily obtained as the same becomes better understood by reference to the following detailed description when considered in connection with the accompanying drawings, wherein:

FIG. 1A is a drawing of an architecture according to an embodiment;

FIG. 1B is a drawing of an architecture, communications links, and processes according to an embodiment;

FIG. 2 is an example relational database organized to keep track of recovery and other relevant data according to an embodiment; and

FIG. 3 is a flow chart of a data collection process according to an embodiment;

FIG. 4 is a flow chart of an analysis process according to an embodiment;

FIG. 5A is a drawing of biometric collection apparatus according to embodiments of the invention;

FIG. 5B is an illustration of collected biometrics data and an example of how the data may correspond and be utilized according to embodiments;

FIG. 6 is an illustration of a user interface according to an embodiment;

FIG. 7 is an illustration of a user interface and map according to an embodiment; and

FIG. 8 is an illustrative flow of a scenario according to an embodiment.

### DESCRIPTION OF THE PREFERRED EMBODIMENTS

Relapse is the single most difficult aspect to manage when facing Alcohol and Chemical dependency issues. One factor in success relates to catching the relapse upfront, preferably prior to a full relapse episode. Various embodiments described herein provide individuals who are struggling with Alcohol and Chemical dependency issues to become more aware in real-time, of hidden dangers that lead to relapse. Such awareness may be based on identified activities, actions, or circumstances from national averages or statistics and/or similar information specifically collected from the individual, or a combination of both.

In one embodiment (which like other embodiments may be a feature or features available from any embodiment) facilities are provided for reaching out and/or mandatory help, discussion or intervention (which may, for example, be court ordered, a rehab requirement, etc.). Such facilities are valuable because the inability to reach out for help, regardless of cause, has the potential to cripple even the most devoted sufferers in crisis and/or difficult circumstances. By collecting various personal, emotional, and situational data points throughout the recovery process, the application (app) (or backend in communication with the application) can track and help identify emotional, environmental, and social triggers that lead to relapse issues. Complex relationships to past known events and outcomes correlated to current circumstances are utilized to provide advance and ‘just in time’ warnings to individuals in imminent relapse danger.

Healthcare providers, emergency response team members, stakeholders, etc. may also be notified as needed and/or as guided by, for example, preferences (which may be mandatory, for example in some treatment programs, insurance reimbursement, etc.). In one embodiment, the individual/user/patient may have the ability or provided permission to alter preferences, which may, for example, be automatically populated based on a selected program. In another embodiment, the preferences are required for entry into a rehab or other treatment facility, for example. The mobile app may be “tied” to the individual via, for example, biometrics (e.g., periodic and/or continual data collection from one or more devices, wearables, implants, etc.) and alerts sent to providers if the associated signals are lost which is then alerted to the healthcare or other provider, for example.

Other data may include, for example, social media and analysis of posts, friends posts (thumbs up, likes, comments etc.), seemingly unrelated data such as electricity use at home, activities at home identified by analysis of electrical signals on power lines, etc.

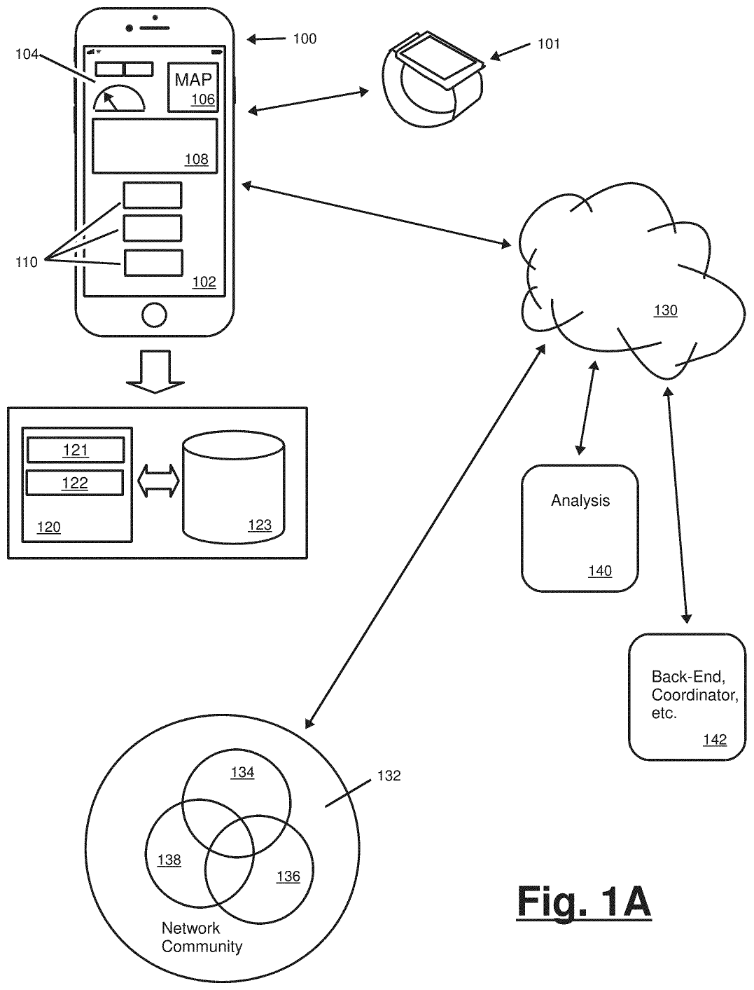

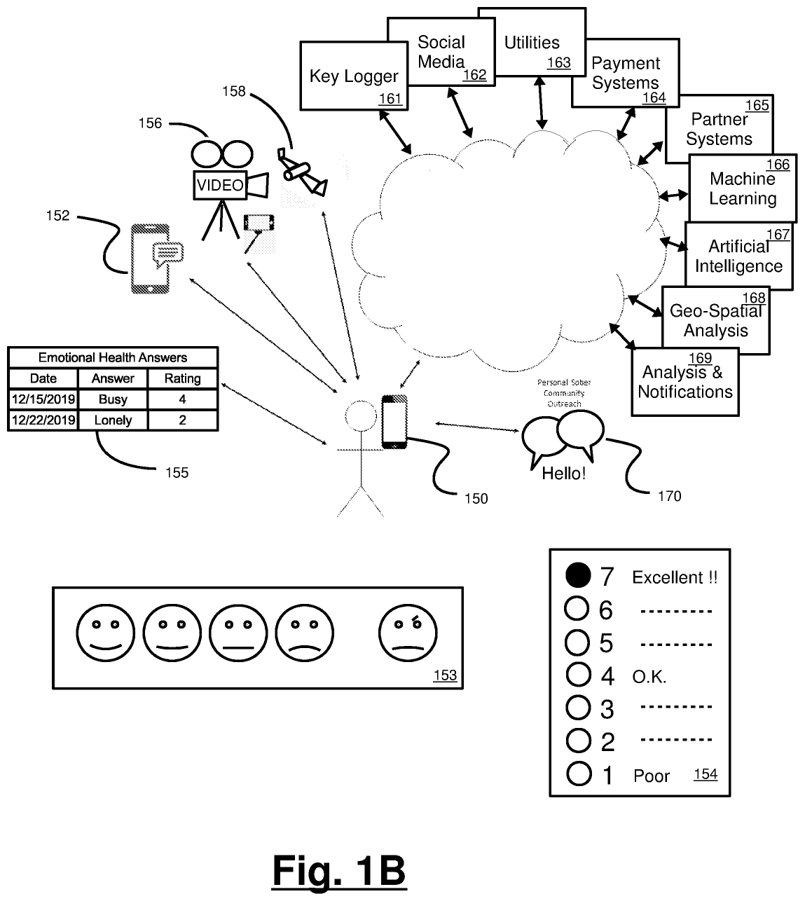

Referring now to the drawings, wherein like reference numerals designate identical or corresponding parts, and more particularly to FIG. 1 thereof, there is illustrated FIG. 1 is a drawing of an architecture including data points and processes according to an embodiment.

The architecture may include one or more features (again no single feature being a requirement), for example:

Mobile First

Designed for ease-of-use. Access your community anywhere on any device. For example, an application (app) on the user's mobile device or access through an Internet terminal, web browser, etc.

Community

The user build's his or her community to support sobriety.

Data-Driven

Based on the latest research, triggers and warnings are identified to keep the user on track (e.g., provide timely suggestions or warnings to the user, notification to stakeholders, etc.)

Customizable

The user may create or establish the settings and community that works best for the user.

Monitors

Keeps track of the user's triggers and environment to help the user get in front of a relapse.

Secure

The individual/user may manage and choose alerts and team members. The data is maintained secure and safe.

A user's mobile device 100 is illustrated with a user interface 102 (as provided by, for example app 121) with a number of optional features. Local processing power (processor 120) executes app 121, and other supporting functions 122 that may be utilized as needed for analysis, communication, or storage, and/or implement one or more features of the app/application. Data storage 123 may be utilized as needed to present the interface, retrieve (and/or process/pre-process) data such as, for example, biometric data from wearable device 101 (or other device, implant, or external equipment observing the user in some manner). Such data may be, for example, galvanic skin response, heart rate, blood pressure, blood oxygen content, electrical or neurological signals, etc. Such data may be pre-processed on device 100 or raw and communicated over a network (e.g., cloud 130) or other communication facility (channels in cloud 130, VPN, etc., for example) to specialized analysis module(s) (e.g., analysis 140) and/or backend 141, for example.

The various features may include, for example, status area 104 which may include an indication of status or boxes for points, scores, and/or dollars—any of which that may be utilized to ascertain completion of tasks, levels obtained (e.g., weeks of sobriety, points accumulated answering quizzes, relapses avoided, etc.) cumulative totals that may be increased or changed over time.

A MAP button 106 may provide fast access to locations of support individuals, standard or pre-planned routes or otherwise highlight other important features (e.g., scheduled locations or activity locations, etc.). A notification area 108 may provide an area for feeds or notifications (e.g., an alert or message from a counselor, community member, or other stakeholder). Buttons 110 may be programmed to initiate one or more features such as taking a quiz, providing a check-in, etc. Such buttons may be highlighted, illuminated, or flashed as a notification that action is needed (e.g., check-in past due).

Various, storage, processing, accounting, and communications may be performed via a network (e.g., cloud 130). Such communication may include, for example, communication with community members 132 which may be a combination of stakeholders including friends 134, medical 136, and/or on-call or reactionary forces 138. Advanced programming or processing features such as AI based or enhanced facial recognition, such as Facial Expression Analysis (FEA) (e.g., mood recognition from a face shot or other photo), object recognition, etc. may be provided by or hosted on one or more remote modules and/or servers (e.g., AI facial analysis, emotional analysis, or mood recognition, hosted on remote server 140, for example). Such programming may include, for example, identification/categorization of photos, objects in photos (e.g., image searches for drinks, tobacco, drug products or indicators thereof), scans of social media postings including image searches and text scanning, etc.

A master program or coordinator (e.g., at Back-End 142) may communicate with the app, user through the app, community, and other programs to gather and further analyze the available data for triggers or trends (e.g., events leading to increased risk of relapse). Such analysis or results may be communicated back to the user in the form of a notification/alert, post on a local feed (e.g., 108), or update of a risk indicator (e.g., 104), for example. Such analysis may be performed by the master program or coordinator at Back-End 142 or performed by specialized routines on one or more remote servers (e.g., 140). The specialized routines may include or utilize API's for various functions related to other platforms or services (e.g., access to social media platforms, retrieval of data from various sources, AI/ML services, etc.).

FIG. 1B is a drawing of an architecture, communications links, and processes according to an embodiment. A user's mobile device 152 runs an app that captures emotional cues from the user (e.g., pop-up query, notification/log-in, responsiveness to queries, etc.). Such emotional cues may be captured, for example, by asking the user to rate his/her current feeling, state-of-mind, or other state. The state may be captured by asking the user to enter a number from 1 to 10, pressing an icon (e.g., emoji) that most closely matches the user's current feeling (e.g., icons 153), selecting a radio button (e.g., button set 154), or other methods. In one embodiment a plurality of sets of selections, for example, four rows of emoji characters each row used to describe different sets of emotions such as, for example 1. General well-being, 2. Anger/frustration, 3. Economics, 4. Control Over Situations/Outcomes, and/or others. Not strictly limited to pure emotions and may include how the user feels about events or circumstances that themselves are not emotions but may, for example, elicit an emotional response or potentially cause actual emotions to crater or peak. The state (or states) may be captured in more than one way and each capture may be used to combine, average or used statistically over time to improve or interpret the ratings. The state(s) may be captured along with other relevant data (settings, recent activities, etc.) and stored in a database, for example (e.g., relational database 154, 200, or other storage, etc.).

Video and/or image responses (e.g., captures) 156 may also be utilized to evaluate the user's current condition, and such video and/or image captures may also be stored and utilized to improve interpretation of later images and/or data captured such as that data captured from the user, the user's mobile device (activities, locations, etc.), or other sources. Such video and/or image captures 156 may be images stored on the user's device, captured at the request of the app, or provided from other sources (e.g., an image from a community member during a recent visit). In one embodiment, the app may request a check-in that includes an image capture, and such image may be compared or contrasted with one or more previous images captured on the same device, posted to social media (e.g., social media 162), or from other sources, for example. The user's willingness to engage the application and complete timely responses (images, questions, or other tasks) is yet another data point.

Location data 158 may comprise, for example GPS signals processed by mobile device (e.g., mobile phone 152), radio signal data from cell towers, or other sources. A user may grant permission for the app or its associated services (e.g., cloud based services) to access location or other data captured or maintained by cellular service or other providers, for example. Such permission grants may be, for example, specific opt-in acknowledgement captured at app set-up, provided in preferences, rehab or court ordered, etc.

Key Logger 162 may be resident on the user's mobile device and/or a service on cloud 160. The key logger may track a user's activities (e.g., key strokes), and may extend to web sites visited, emails read, application utilized (including input/output from such applications, websites, etc.). The extent to which the key logger 162 (or other programs/processes described herein) extend(s) into various areas of privacy concern, permission to do so may be provided, for example, by specific opt-in acknowledgement captured at app set-up, provided in preferences, rehab or court ordered (e.g., terms of entering rehab or release, etc.).

A social media module 162 may include facilities to monitor and scan social media posts. Posts by the user, the user's friends, contacts, and those the user comes in contact with. Such posts and analysis of the posts may be compared in real time with other data collected to confirm a user's current status or state-of-mind. For example, a user checks-in and provides a self-assessment including a picture, such picture may be compared to the self-assessment score and a comparison of previous photos including recently posted pictures, for example. Such a series of photos may show a changing emotional state that may indicate a level of intervention or other measures to assure the user stays on track in their recovery.

Utilities 163 may include capture and use of telephone, electricity, or other utilities that may provide data reflecting the user's activities or lifestyle. Such data may be captured, stored, and/or processed (e.g., at Back-End 142), and, for example, ultimately used to verify normal or changing patterns of activity or habits of the user. For example, a user that typically maintains a bedtime of 10:30 PM suddenly shows late night or early AM spikes in electricity use may indicate that a welfare check, emotional cur update, or communication from a counselor may be in order.

Payment systems 164 may include credit card, debit card, Pay-pal, Zelle, Venmo or other services in which the user has granted permission for the app (or its associated backend or other services) to access financial information such as purchases and payments. Such data may be compared to other events/data and/or automatically notify the system of a suspect purchase.

Partner systems 165 may include any other systems or collectable data (which may have a similar origin to systems described elsewhere herein) regarding user activities or the user's physical or mental condition (including medical data, club data, golf scores, gym/workout data, biometric data, internet usage/screen time, Netflix or other accounts information, etc.). Machine Learning module 166 may have access to data collected by the app and/or data from all other systems (each again with appropriate permissions).

Geo-Spatial Analysis module 168 may provide analysis of the user's location. For example, identifying the user's specific location and a probably activity at that location (is the user in a store, is the user in a liquor isle of the store?—a red flag alert warranting an immediate contact or intervention).

The machine learning module 166 may be set-up to search for patterns in the data including patterns applicable across the user spectrum, specific classes of users (e.g., alcohol issues, tobacco issues, drug issues, gambling, gaming, for example), and for the specific user as an individual. Such patterns may include, for example, identification of events or patterns of conduct across one or more partner or other systems that may identify a changing mental state, changing living conditions, changing habits, etc. Such changes may include, for example, identification or indications of a potentially more depressed state and/or a more elated state, each of which may reveal a situation or circumstances where an intervention or other contact with the user is warranted.

The machine learning module may vet rules by running them against large data-sets collected over time (e.g., data-sets related to the user, and/or larger datasets such as a class of users, and/or all users). Such rules may be weighted based on, for example, an accuracy probability for each class of users and for this particular user. Such rules and/or their weightings may then be utilized by Artificial Intelligence (AI) module 167 to make decisions regarding a user's state or probability of a relapse and/or need for intervention or other contact.

Analysis & Notifications module 169 may provide programmed notifications based on pre-determined events and/or notifications based on analysis performed by one or more modules (e.g., Artificial intelligence/Machine Learning modules 166 and 167), and such analysis may result in, for example, identification of an issue (e.g., serious change in the user's emotional state, root cause identification, etc.). Notifications may include, for example, pop-up requests for emotional cues, request for specific information from the user (are you on track today?), notifications corresponding to analysis, and/or a notification to a counselor or other support member which may include contacting support members (e.g., network/community) based on level of experience and/or proximity to the user.

Such notification may include all the particulars of the user to be contacted, including name, photo, location, and fast access buttons or highlighted fields (e.g., phone number) to make immediate contact, and such notifications may include suggest approaches or contact methods (e.g., “user more likely to pick-up if you text first”). The counselor or other support member (e.g., personal sober community outreach 170) may be tasked with making a personal contact (e.g., phone call) and then possibly included in a physical/actual intervention group depending on whether the user can be reached and/or responses received).

Such activities and any escalation/follow-up may proceed according to a protocol that may be organized, arranged, and implemented by the system (e.g., Back-End 142 or Analysis & Notifications 169, for example). Other modules, such as programs resident of the user's mobile device may be programmed to participate or coordinate such activities as well.

One or more analysis may conclude a risk level that is elevated but not imminent, and in such cases, automated messages or interactions with the user such as to notify the user about the elevated condition and/or look further into potential issues without immediate or direct support member contact may be utilized (e.g., low level issues). And the same or similar app/user interactions and messages may be utilized in conjunction with team member notifications, communications, and/or mobilizations. Team member notifications (including team leader assignments), group organization messages, etc., may also follow a protocol that, for example, requires an affirmative response accepting responsibility for taking necessary actions in response to the notification—absent which the notification may be re-routed to other team members (or other groups/teams/contractors if none are available) until the appropriate team or team member is identified and ready to respond.

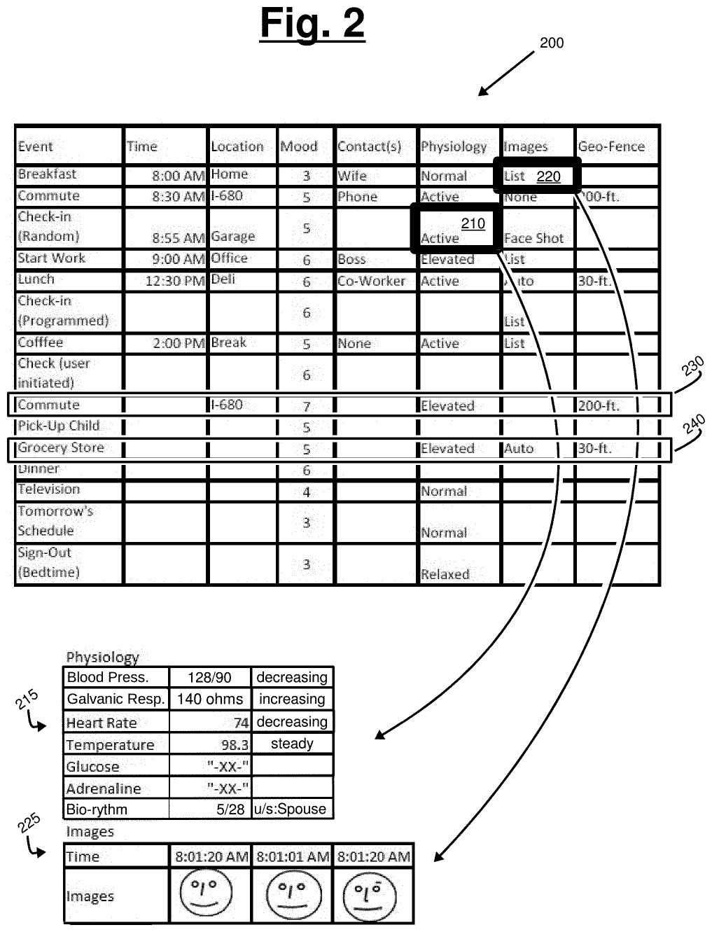

FIG. 2 is an example relational database 200 organized to keep track of recovery and other relevant data according to an embodiment. The database may include various data points, such as, for example, routes, frequently visited stores, etc., list of known danger spots (bars, dealer locations, etc.), purchase histories, biometrics, and other data which may be correlated or used to correlate against other factors including use of the same to determine a probability or potential likelihood of a relapse or other event. One such correlation may be, for example, recognition of an event or conditions that could trigger a relapse which is then prioritized for a response.

Such response may be an interaction with the application, a direct call from a healthcare professional or friend, and/or an immediate response team intervention which may be in the form of taking the individual out of the situation by force if necessary (non-revocable and approved in advance, for example). Other responses may include stand-off observation but ready to intervene, for example. Such responses utilizing team leaders, team members, or others (e.g., contractors) may be organized by the application (or backend) by, for example, first sending notification to the team leader (or selecting a team leader, e.g., via the above discussed protocol), and then filling the team as needed (e.g., as suggested by the application or backend, or as specified by the team leader) using a similar protocol.

In the example database 200, a subset of potential data is shown, including time/location stamps, Mood indicator (which may be a predicted mood or answer from an emotional cue), a contact if applicable (e.g., who is the user interacting with at this time/location), Physiology, Images (which may be application requested images or images taken automatically, and geo-fence data (e.g., is the user near, approaching, or on the way to a geo-fenced location (e.g., liquor store) or location having a geo-fence (e.g., Safeway—O.K. to enter Safeway, but beer and wine aisles are geo-fenced as impermissible to enter).

The database may include parameters of each user's geo-fencing bounds as some users may be held to more stringent geo-fencing policies (e.g., in some cases entering Safeway may not be permissible even though aisle level geo-fencing is available). Portions of the database, such as geo-fencing parameters may be copied and stored on a user's device and interact with GPS signals or locations to produce pop-ups, notifications, physical alerts (e/g/, phone vibrations), sounds (e.g., alarms—including, for example, loud tones that are not user configurable and may only be turned off by exiting the geo-fenced area). The geo-fence violation itself is logged into the database and may, for example, be sent via alert to the user's counselor or a team leader and may be the basis for formation of a response team. Geo-fence violations, like other events noted and/or stored in the database, may then be compared or correlated to other data collected in or around the same time, such as one or more physiology factor, emotional state, etc. Such comparisons or correlations may be done with the aid of ML/AI module 166/167 and provide a basis for future predictions, alerts, or warnings.

Various entries relate to other tables or databases, such as, for example, physiology entry “Active” 210 (a summary of physiological data) points to physiology table or database 215 which may provide, for example, a more complete picture of the user's physiology at the date/time stamp. The physiology table may include blood pressure, heart rate, temperature—and other items that may be determined from sports monitors or other equipment (e.g., medical equipment at a doctor's office), watches (e.g., Apple watch), gym equipment, and other devices). The physiology table may include an entry for biorhythm or stage of a hormone cycle for the user or that of the user's partner. The physiology (as a whole or individuals data points) may then be correlated to other events at/around a same time, and such correlations may be used in future predictions or indicators of emotional health, for example.

In one embodiment, the physiology table includes entries from EKG, ECG registered devices and/or galvanic skin measurements (which may be part of external devices such as watches, rings, or electronic patches, or may be an implanted device, for exmaple).

Images entry 220 may be an image, data about an image and/or a list, for example, of multiple images and/or data. Such images may have been taken in response to a query from the app (e.g., emotional cue query that included a request for one or more snapshots). The various images may be time stamped and stored for analysis. Data about the images may be extracted from AI routines or services that note significant messages, tones, or emotional content of the images and stored (and then used in any contemporaneous or past event evaluations, for example).

The database as a whole stores this and other data to be processed and reviewed for patterns and other information relating to the user's emotional state and potential for a relapse. Looking at the database as a whole, and objectively, it can be determined, not knowing any other information about this user, that a more likely time for an event appears to be at the Commute entry 230—which shows a combination of elevated mood, no contacts, elevated physiology, and relatively close proximity to a geo-fenced location (200 ft.). Without knowing more, it may be prudent for the system to send a notification or alert to the user's support network for an outreach. Grocery store entry 240 (not long after the commute entry) includes some of the same potential warning signals/combination.

That being said, such analysis is likely more meaningful when considered in context with a history of such entries collected by the app over time. The history may show such events have occurred under similar conditions without impact and may (at least at this time) be set-aside (but may still warrant a follow up email telling the user s/he successfully navigate a rough commute or potentially dangerous store event. Alternatively, the history may show past relapse events under similar conditions warranting an immediate and direct intervention.

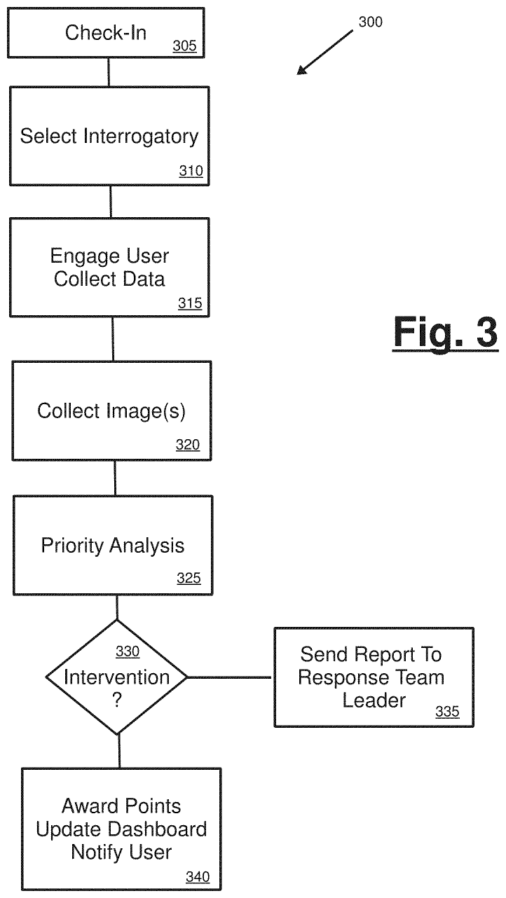

FIG. 3 is a flow chart 300 of an exemplary data collection process according to an embodiment. The data collection process may take the form of collecting biometrics, travels, events, and/or emotional cues. For example, at step 305, a check-in is initiated. A interrogatory may be selected which is, for example, any of a request that the user rate his/her emotional level or mood on a scale of 1-7, select a emoji reflective of their mood, pick a color that describes how they are feeling, or others. The interrogatory may be randomly selected, may be consistently selected for a period of time (e.g., same query for a week, then switch to another query), or may be set to a query that user most favorably relates to. The selected interrogatory is then used to engage the user and determine their emotional level or mood or current mental condition (step 315). The interrogatory may include the collection of images (step 320) that may be taken by the user. In one embodiment, images are acquired by commandeering the user's mobile device camera and actively looking for a face shot of the user and capturing it when it is available. Such a routine may be utilized to collect face shots at other times which may relieve the user from that duty at least at times when it would be inconvenient to capture an image manually. All such images may be processed with FEA or other techniques to determine the user's current state (emotional state), and then, for example, stored in the database along with the determined state or other metadata. The determined state may also be supplemented, modified, and/or cross checked against other contemporaneous data such as location, recent activities, biometrics (e.g., vital statistics, GSR, etc.), compared to previous emotional states of the user (and previous contemporaneous data), and compared to similar data of other users, for example.

The information and images collected may be analyzed to determine the user's current emotional level or mood or mental state. Such analysis may include comparison to biometric data collected as part of the check-in and/or review of biometric data compiled over time, and/or review of biometric data compiled over time under similar circumstances (e.g., reviewing previous times the user has been at/near the user's current location and comparing to the user's biometrics at those times to current readings). Such analysis may be, for example, a priority (step 325) or real time analysis to support a decision (e.g., decision to intervene—step 330). If an intervention is warranted, a report or notification may be sent to the user's sobriety network outreach community (e.g., Response Team Leader (step 335). The notification may be managed by the system within the user's network to avoid conflicts (e.g., reach out to individuals in the user's network that are appropriately trained and available at that time, for example). If a selected or nearest trained member (e.g., team member and/or team leader) is not available (e.g., no response, or declines), a next most qualified or next nearest member (or members) may be contacted.

In the various instances where a team needs to be assembled the team leader and team members are contacted by the app/backend noting the particulars of the situation, user's history, etc. If the team leader (or other member) is available, a positive response from the team leader (or other member) acknowledges receipt of the notice and acceptance of the responsibilities of being part of the team. The team leader is accepting management of the team and committing to taking action. The app/backend handles many of the traditional team management functions (e.g., sending team messages, collecting/organizing responses, preparing team lists, managing calls/video conferences, etc.) so the team leader can spend more time preparing and planning for the necessary steps contacting or intervening for the user's benefit and insuring the response follows established procedures for the intervention or other task.

In one embodiment, the team leader is asked to provide additional information as to the plan of action for any response. This may be, for example, noting that a group call is being organized, or that an intervention is planned, and/or both, for example. Some standardized actions may be selected on a menu such as call, organize intervention etc., and such selections may, for example, fire an API trigger to another system or module (or a disparate system) to handle that task or portions thereof (e.g., find and organize volunteers for an intervention). Such API may be, for example, a process outside of Prelapse (the app) and may be hosted by another entity altogether.

In this manner the system initiates and helps manage the response team to most effectively and quickly provide an appropriate response. In cases where no qualified members are available, a volunteer may be contacted to intervene along with phone support from a qualified individual. Such interactions may be recorded on the user's device (and the volunteer's device(s)) for later review, discussion, and process improvement. In one embodiment, if members of the user's network are not available, trained volunteers (or paid response teams) outside the user's network may be contacted. Thus, the system may automate and manage contacting volunteers/personnel in a tree-like hierarchy to assure that the user gets the most qualified contact/intervention available as needed to prevent a relapse or lessen the impact of a relapse in process as soon as possible. And of course, not all interventions will require larger manpower and lesser event may be managed, for example, with a phone call from a therapist, case worker, or other individual.

The system may further facilitate collection of information regarding the user's emotional state by requesting information about the user's emotional state from those with who the user interacts, including, for example, those in the user's sober network, friends, co-workers, health professionals, team members (e.g., requesting an evaluation after an intervention), and others. The system facilitates collection of the emotional information and also facilitates interactions with the user, such as interventions or in-person check-ins (scheduled and/or random) if applicable (e.g., part of a user's rehab release plan, order from a doctor, therapist, rehab center, or court, etc.)—all of which may include a follow-up from the system/app to the team member or other professional, etc., requesting an evaluation or impression of the user's emotional state at the time of contact. Such evaluation may then be compared to information that the app has collected in/around the same time frame and predetermined time periods before and/or after the contact to provide additional insight as the user's emotion state and probability of relapse or other event.

At certain times, it may be appropriate to award points or suggest the user make a diary entry (the system may include facilities to quickly add diary entries (e.g., automatically make a suggested diary entry if accepted by the user) (step 340). Such points may, for example be added to a total points maintained on a homepage or dashboard of the app and provide a form of reinforcement.

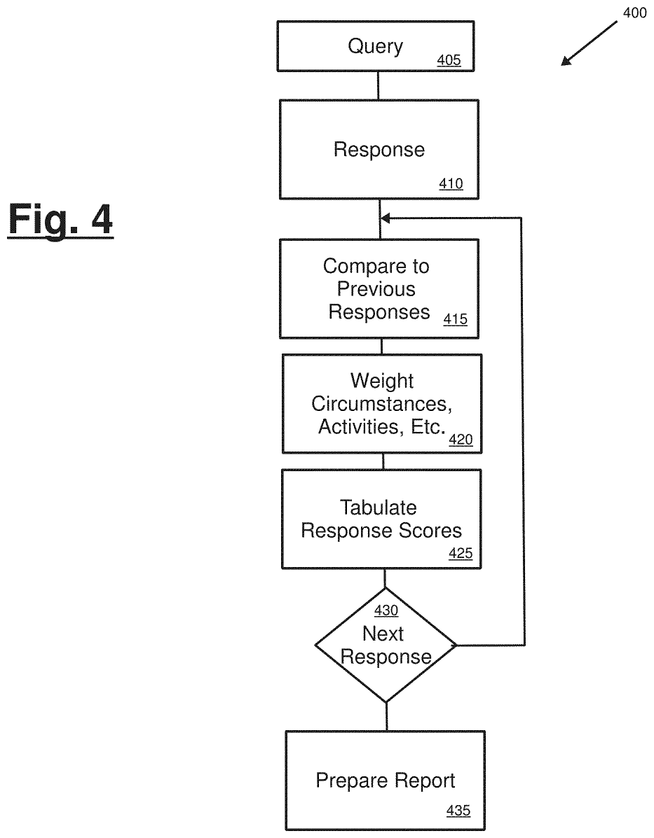

FIG. 4 is a flow chart 400 of an exemplary analysis process according to an embodiment. The process may be set up for analysis that may include, for example, identifying events or potential events, and issuing notifications/alerts to the various stakeholders as, for example, enumerated in a preferences function of the application or built-in by design for a program. The process may begin by providing a question, quiz, puzzle, or other query (step 405) and capturing a response (step 410). The response is compared to previous responses to the same, similar, or similar types of question or query (step 415). If the answer is significantly different from previous similar queries, a red flag may be raised for further analysis/query. The response may also be weighted based on, for example, known circumstances, such as recent activities, biometrics etc. (step 420), and a cumulative score or total may be maintained (step 425). The queries or other assertions/responses may continue (step 430) and associated comparison/weighting/tabulation until complete and a report or score may be provided to a decision module (e.g., step 330, FIG. 3).

Accordingly, the various embodiments include multiple layers of data gathering capability, some that require a user's permission—others that are publicly available (such publicly available data may include social media, local and national news cycles, etc.), and such data may be organized and analyzed (e.g., as described herein or in other ways) to establish relationships and patterns with respect to the various data points. Further, such data may also be collected from other individuals in the user's various circles, and particularly those whom the user interacts with on a regular basis (but may certainly include others that interact tangentially or more remotely). For example, data may be collected on additional individuals is from those in the user's sober network and at all levels of influence.

The user's sober network may include those identified by the user while using the app, and once linked to the user may be sent a download link for the app (or an adjunct data collections app) along with permissions including opt-in for any collections that may be desirable. For example, social media posts, location tracking, and face shot analysis—a similar or subset of data collected with respect to a user may be collected on/about individuals in the user's community, sober network, or other groups that, for example, interact with the user. This data is then analyzed/searched for patterns with respect to the user (including FEA of the user and the other individuals), the user's activity patterns, and emotional state, which may ultimately reveal patterns or circumstances that are related to or that potentially affect the user's emotional state, identify triggers, etc.

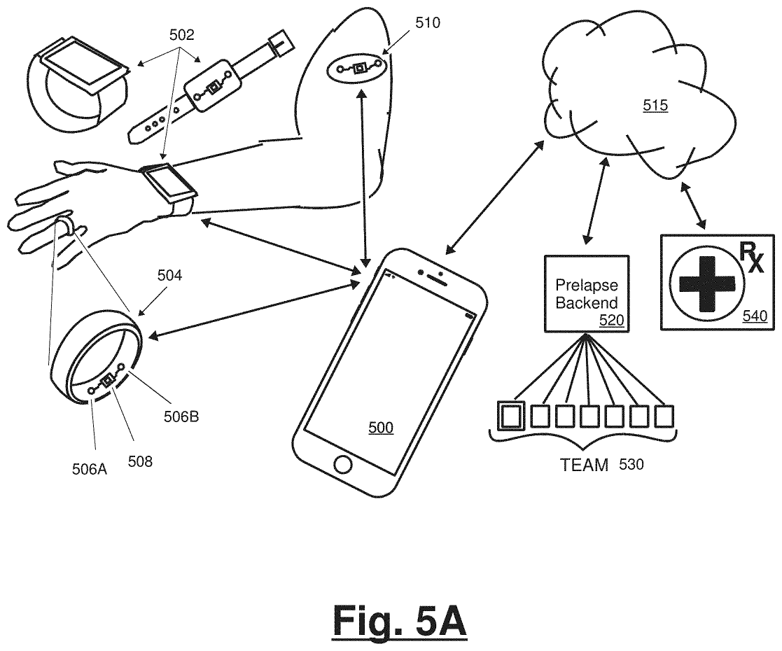

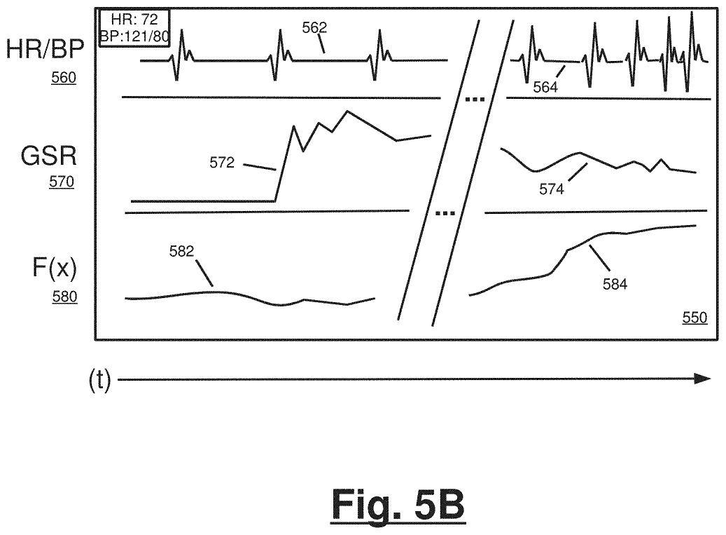

FIG. 5A is a drawing of biometric collection apparatus according to embodiments of the invention. A wearable device (watch form factor) 502, a ring 504, and/or a patch 510 may be worn by a user. The devices include apparatus for measuring biometrics such as heart rate, blood pressure, galvanic skin response (GSR), and others. The device may include, for example, a plurality of electrodes (e.g., 506A and 506B), and a processor/communication module 508. Other hardware including LEDs, photo-sensors, RF, and/or audio capabilities may be included as desired to implement one or more biometric measurements and communication with the app (e.g., installed on mobile device 500). The electrodes, may, for example, be configured to measure resistance and the processor may be configured to calculate GSR along with any other biometrics the device is configured to measure.

The wearable devices may be placed in other locations, and implantable devices may replace or supplement any measurements (e.g., an implanted ECG, which may have been implanted specifically for use with the app or for other medical reasons, the data from which may be accessed and forwarded to the app on mobile device 500). If an implanted device has other medical functions, the app my facilitate transfer of the data collected to a medical or RX facility 540 via cloud 515, and/or a physician with knowledge of the user's Prelapse monitoring may request access to all data which may also be forwarded to facility 540. Further if the user already has a monitoring devices, external or internal (e.g., implant) for other purposes, with appropriate permissions the data from existing devices may be collected an forwarded to Prelapse.

Facility 540 may be a rehabilitation (rehab) center or program which receives regular updates and maintains current status on a plurality of users and has access to any of the users' data on the backend 520, mobile device 500, and/or other devices. The data collected is ultimately placed, for example, in a database (e.g., relational database 154, 200, or other storage, etc.) and used to calculate a current emotional state of the user, probabilities of relapse events, and otherwise facilitate decision making related to features of the app and its associated processes (e.g., preparation and organization of team 530, when needed).

FIG. 5B is an illustration of collected biometrics data and an example of how the data may correspond and be utilized according to an embodiment. The illustrated biometrics comprise a subset of data that may be collected and are provided as an example of the type of data collection, correlation, and processing that may be implemented. Heart Rate & Blood pressure data 560, Galvanic Skin Response 570, and F(x) which may represent any of data or combined data collections, and here specifically providing an example of a combination of all other data collected and processed to determine the current emotional state of the user and risk of relapse but excluding GSR and HR/BP for purposes of this example.

As can be seen in segment 562, a user has a steady and normal heart rate. In a same time frame, the Galvanic Skin Response 570 has a significant change (increase) 572. Such an increase may be, for example, an increase in resistance measured at the surface of the skin (other measurements may be utilized), for example as measured between electrodes on an appropriately configured watch, ring, patch, or an implanted device. The increase may be, for example, from 150 ohms to 250 ohms, or a percentage jump of, for example, 50% or more, or a steep jump in GSR from any level—any or all of which indicates that the user is responding to some stimuli which may be meeting someone, being startled by an event, and/or passing by an outdoor restaurant and smelling alcoholic drinks (even if not consciously aware).

Like other data collected and monitored, significant changes are important for the app to know the circumstances causing the change. All other collected data at about the time of such changes (e.g., location, other biometrics, activities/exercise, sleep/work/eating cycles) may be flagged as a set of data representative of a significant change. The change may also trigger an automated response from the app (or call from a team leader) that may, for example, ask the user what his/her current situation is.

It is important to note that in the same time frame, the user has a normal heart rate 562 (heart rate has not yet reacted significantly to whatever the stimulus was), and the F(x) segment 582—processed data from all other data collected shows only mild variations indicating all other factors appear reasonable.

Moving ahead in time, the GSR begins to recover 574, but all of the other factors F(x) 584 have increased significantly along with the user's heart rate 564. In fact, the user's heart rate and other factors clearly indicate the user is experiencing some sort of turmoil and may likely be in an emotional state conducive to relapse. Such conditions trigger an immediate app response and formation of an intervention team.

An important aspect of the above example is that the GSR showed potential of the turmoil before the other factors and other biometrics kicked in to indicate that turmoil. The GSR may have responded before the user was even conscious of the potential turmoil. For example, alcohol odor wafting from a restaurant that may be sensed but faint enough that the user does not consciously recognize it, the GSR will react (at the sub-conscious level). Accordingly, along with conscious reactions, embodiments of the present invention take advantage of the sub-conscious reaction shown in galvanic skin response which is a data point from which to evaluate a user's emotional state and to correlate with other data for current and future emotional state predictions.

In this example correlation may include, for example location—the location of the user at the time of the GSR response may be noted as a potential trigger point and evaluations of the user's state may be more critically evaluated when passing this location in the future. Warnings/Notifications to the user and/or others (e.g. team leader) may be issued when the user is on a route that may include the location or similar locations. Maps and routing as provided elsewhere herein may take the location into account. A geofence may be established around the location. Other sensing devices may be placed on high alert or threshold levels reduced when near the location (more likely to issue team/team leader notification).

Correlations to other data may also be made and appropriate notifications when similar conditions occur. Accordingly, such correlations may be made with any of the individual data points or any group of data points including any of the data points discussed here and/or other data points that provide any information whatsoever in determining a user's state of mind, emotional state, probability of relapse, etc. (including data points with respect to such states of family, friends, and associates of the user).

Further, the GSR data collected is maintained and along with the other data points provided to Machine Learning (ML) and Artificial Intelligence (AI) routines to correlate the data in every possible way and look for patterns across the dataset. The GSR being a response that can provide an early indicator of downstream stress or emotional conditions conducive to relapse potential and a key to identify such conditions before a full-blown relapse occurs.

It should be understood that the illustration of FIG. 5B is just one example of a possible GSR and other data of an individual user during essentially one time period. Data collections at the same time but different location (or visa versa) under similar circumstances has a probability of a similar result but could be very different. Accordingly, the collection of data over longer time period will likely increase the possibility of correlations of current data providing better information. This includes correlations over the larger dataset of all users which may be applied to any specific user, as well as the user's specific data.

Further, the collection of data including GSR for a large number of users increases the data pool to the extent average responses to known conditions usually has at least some correlation to current events for any particular user. Further yet, the differences between the general population response (e.g., emotional condition) and an individual itself can be statistically meaningful when compared to an individual user's emotional status a relatively few number of times and can quickly identify triggers when the user's status changes significantly.

GSR measurement may also be specifically noted when the user is performing actions of known quantities. For example, measuring GSR while the user is taking an emotional que questionnaire (e.g. selecting feelings, answering questions, etc.). Veracity of the answer might also be implied (e.g., if all other conditions point to low emotion state (high relapse potential), and the user responds that s/he is happy and things are going well, but the GSR indicates agitation (especially if such responses were confirmed earlier) then it may be appropriate to provide a further inquiry and/or initiate a team response.

The GSR may also be measured at precise points of any particular activity, such as, for example, while taking a selfie, texting certain individuals (permissions may be set such that Prelapse reviews who the user is communicating with (e.g., text, voice, email, conference call, zoom, etc.), and the GSR may be evaluated for how well the user is reacting internally while interacting with these individuals. Such reactions may be mapped to each line of text as drafted, sent, and/or received/read.

The app may recognize an in-person conversation via an open mike with a voice identifier running, or the app may ask the user to identify a conversation during or after it is occurring (which may then be used to recognize subsequent conversations with the same person). If such interactions are consistently negative (e.g., as mapped against GSR or other biometrics) appropriate counseling for the individuals and/or pair may be suggested by the app, for example, suggested to the team leader who may then discuss with the user.

Other biometric data may be similarly utilized and correlated with other events. Types of biometric data such as breathing rate, muscle activity, brain activity, activity of different portions of the brain, or any type of physical or neurological metric, from any type of sensor (e.g., muscle sensor, EEG sensor, etc.) that may capture that activity/metric may be similarly utilized as discussed herein with respect to GSR or any other biometric.

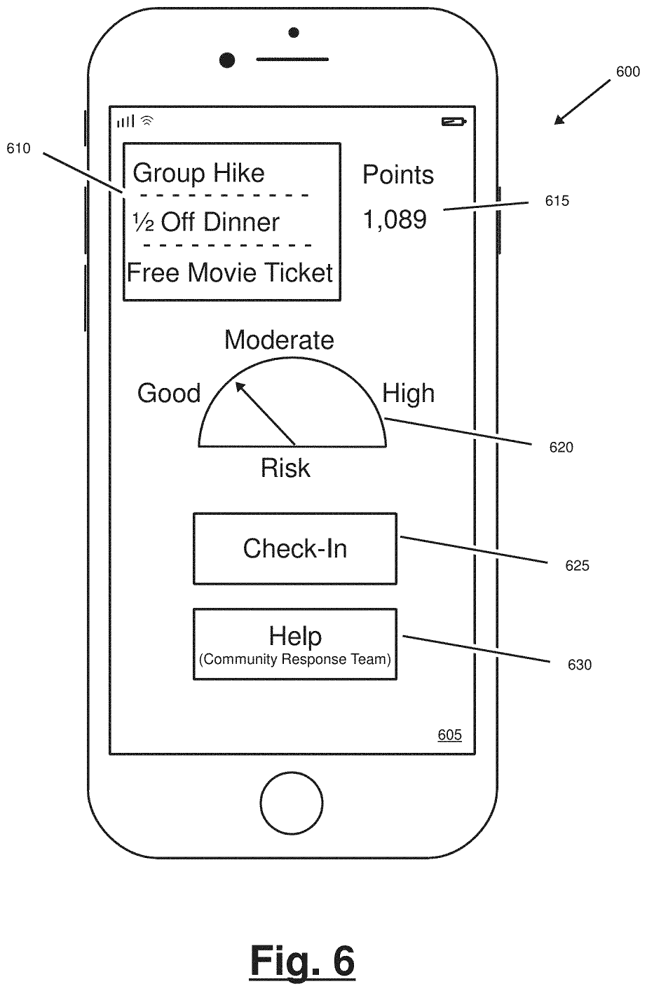

FIG. 6 is an illustration of a user interface 605 according to an embodiment. A user's mobile device 600 hosts an app that displays the user interface 605. The interface hosts messaging summaries which may be, for example, event notifications (e.g., group hike) and specials (e.g., dinner discounts, movie tickets, notation of point awards, etc.). Such specials may be, for example, awards for reaching certain milestones, gifts from groups supporting recovery, etc. In one embodiment, such notifications appear through the mobile device's messaging system as set-up in the mobile device's preferences.

A points area 615 displays the user's current points total, which may highlight recent awards. Such awards may be granted from a pool of award points for successful completion of milestones, check-ins, maintaining a diary, etc. A risk meter 620 may provide, for example, an indication of a user's current risk profile, an indication of the user's current emotional level or mode or mental condition, or other evaluable condition or state. In the case of a risk profile, the app or system (e.g., Back-End 142) accesses the various conditions and trends currently affecting the user to produce the risk profile—this provides the user with a feedback mechanism from which can help the user judge their own current condition and add an extra metric to any decisions about activities. In one embodiment, tapping the risk meter (or another icon, drop down menu, etc.) brings up data or a mini analysis that may show, for example, a set of data points (e.g., most influential data points) from which the risk factor was determined. For example, data points from the relational database, trend analysis, etc.—providing additional feedback, information, and explanation to the user or their respective sobriety network.

Check-In button 625 may be utilized to respond to an app generated request for a checking, or for a user-initiated check-in. A help button 630 may be used when the user feels conditions warrant an immediate reach out to his community (e.g., 911 to user's support community). The button may initiate, for example, a call, video conference, etc. with one or more team members who will interact with the user to discuss the current situation and provide a plan to address any difficulties. In one embodiment, at the same time, a response team may be notified to make a physical intervention if warranted. A warranted intervention may be determined by team members on the call, or other protocol (which may be automated based on circumstances such reported location, risk factors, contract with user, etc., for example). Team members on a call may communicate via a specialized interface that allows them to, for example, suggest an intervention by pressing a button which then automatically asks the team leader (and/or other team members) (e.g., via notification on the specialized interface) if they would concur or support intervention.

Such suggested intervention may, for example, be privately shared between the suggesting team member and the team leader or with the entire response team (e.g., pop-up notification on team member device: “Team member George suggests an intervention”). The suggested intervention may be considered privately or discussed on the call at the team leader's discretion (e.g., depending on circumstances).

The response team may be initiated by an automated message to one or more team members (not necessarily on the call) with, for example, particulars such as location of the user, brief/recent history, and a link to the video call feed. The response team may be composed via automated computer selection based on, for example, from a pool of available team members in order of, for example, experience, proximity, current situational knowledge, etc. Substitutes may be automatically selected, notified, and verified in response to first round selected team members who are either unavailable or non-responsive.

A user interface according to an embodiment may provide facilities for, for example, data feeds and communications with processing for maintaining event logs, access to self-help, preferences, social network feeds and links. Separately or additionally, schedules may be applied, for example, to guide the individual through a routine or daily activity and provide easy check off and suggestions to modify or organize behavior, for example, to reach a next task completion or milestone. Such task may be, for example, completing work, grocery shopping, and/or going directly home.

Such tasking may include a shopping list for a suggested meal and recipe for the evening's meal (or a suggested take-out, that may be, for example, guided along a preapproved drug/alcohol free route). Such tasks may be linked to social media posts as required by the program (e.g., on Facebook or local recovery page). Posts made are also data points recorded and used to analyze current and future activities/conditions. Such task may be general in natures or provide specific instructions or even micro-management of the user's activities (which may depend, for example, on the user's experience, fluidity, or level of acceptance of the program/app). For example, a user's trip across town may be provided with a specific route and specific way points, and, missing a scheduled waypoint or destination time frame or route may result in a notification to team members.

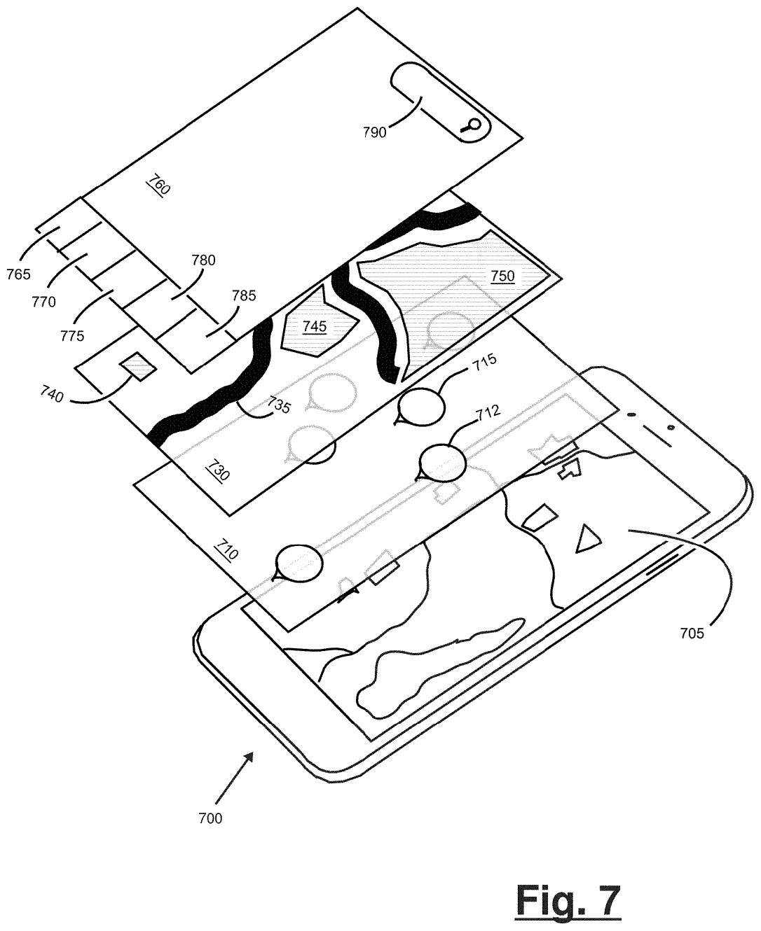

FIG. 7 is an illustration of a user interface, map, and other features according to an embodiment, on, for example, a user's mobile device 700. A map 705 provides a base layer of the user's surroundings. Overlay 710 may include locations of friends, contacts, community support points, places of solace or meaning to the user may be provided with markers (e.g., markers 712/715, for example). Such markers may be customized by the team leader or licensed therapist, especially initially, and controlled until early or other stages of recovery having higher relapse potential are past.

Such markers may be vetted via AI/machine learning routines to ascertain an appropriateness of the marker. For example, certified or trained support members would likely pass vetting, contacts may pass subject to additional vetting designed to weed out potential undesirable influences such as those that might be enablers or not fully in-tune to the user's situation. Liquor stores would most likely be excluded, as would locations of previous re-lapse or breakdown events, for example.

Such markers may include any of icons, photos, graphics, or text associated the person/contact being marked. Such markers may, for example, be touch sensitive to provide immediate team or support structure communication. Overlay 730 may include geo-fences or outlines of forbidden zones or off-limit areas that allow the user to plan a route that does not include things such as, for example, known bar neighborhoods, areas of problems for the user, known drug alleys, etc. For example, overlay 730 includes OK route 735 and off-limit areas 750, 745, and 740. Such forbidden zones may be determined based on known neighborhood characteristics (e.g., an area known for nightlife/bars), known previous issues with the user (e.g., location of a previous relapse event), or data collected with respect to the user that was negative whether or not the cause was determined (e.g., an area that consistently causes a high GSR response when passing by).

Top overlay 760 includes access to application (app) features such as search 790 (e.g., search for support or community members/resources), Location 765 (e.g., centering map on the user's location), List 770 (e.g., populates the map with markers representing the user's support team/facilities, or the user's customizable list of people or locations, for example), Favorites 775 (e.g., user's favorite or memorable locations on the map, for example, or, links to other platforms, web browser, social media, etc.—any of which may be monitored or used for data collection for other functions of the app, for example), Circles 780 (e.g., selectable sets of various contacts, support group, work group, clubs, etc.), and profile 785 (e.g., set up the user's profile that may be accessed by friends, contacts, team members ,etc., the same profile may be utilized, for example, to populate profiles on other apps such as Facebook, twitter or others, and again, may also be subject to monitoring/data gathering for other application functions, and such data gathering may be by agreement (e.g., opt-in agreement) with the user, court order, and/or permissions granted for the user's various accounts, for example).

Accordingly, various embodiments may be very sophisticated and a user's app may be set-up by a rehab facility according to conditions of re-hap, court-order, or under a general program (e.g., community or group funded or via insurance) which may include cash or point awards. Such programs may include pre-arranged persons of responsibility such as team organizers, response team members, etc. Such team and/or response members may be part of the user's existing sober network or other stakeholders in the user's recovery. Alternatively, such team members may be composed of paid professionals who would then authorize or advise on bringing in the user's existing network on a case-by-case basis. In yet another embodiment, no network, no insurance, and/or no rehab is utilized or available and the user may download the app on their own and use it as-is without additional outside help. In yet another embodiment, the user may opt for paid services through the app, for example, when at a high risk or low point emotionally, the user reach out by (or app may suggest) spending $50 for a counseling session with counselors that may be contracted by the application.

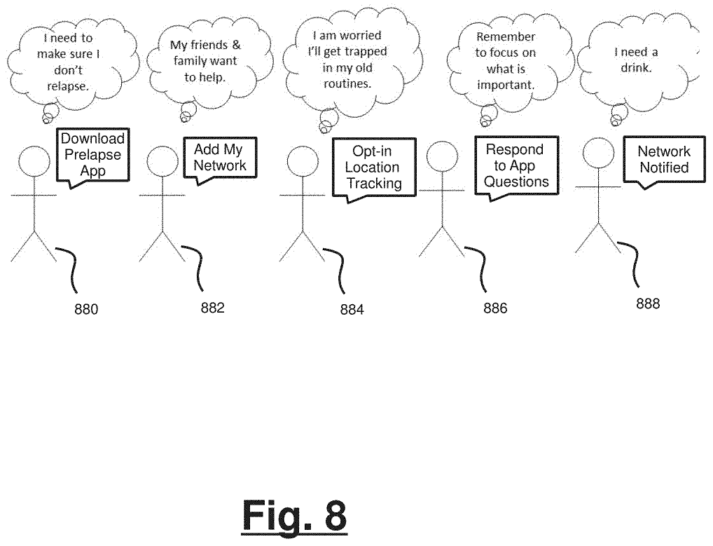

FIG. 8 is an illustrative flow of a scenario according to an embodiment. An individual user or patient download's the app (application) to his/her phone, personal device, or laptop, for example (step 880). Any internet appliance may be utilized, and, depending on the individual's personal situation, such download and operations discussed herein may be performed by a handler, secretary, personal assistant, etc., however, please note that some embodiments may include questions, games, quizzes, or other activities that are designed to be part of an overall recovery and treatment and should, for best effect, be performed by the individual).

Once downloaded, the app may prompt the user for relevant information, which may include, for example, answering a list of short multiple-choice questions. The questions may be designed to evaluate the individual. The evaluation may be, for example, to establish an emotional baseline. The evaluation may be, for example, to determine if a crisis or other emergency situation exists right now, at this moment (which may have been why the individual downloaded the app at this time, for example). In one embodiment, the app can determine its own baseline over time through use of the app. Even if just left running in the background on the user's device—a baseline can be established through collections of events and activities—though less timely than if the individual establishes it from the beginning. Time is of the essence as preventing relapse because it is a most dangerous event for the individual, stakeholders, and non-associated individuals that may just happen to be nearby, so the user may be pressed on this point, and in one embodiment, failure to establish a base line does result in a personal call or contact to explain the reasoning and why it is important.

A next step establishes the individual's network, health providers, etc. For example, the app may direct the individual to input his/her sobriety or support network (e.g., step 882). This could include but is not limited to Parents, sponsors, therapists, life coaches support groups etc. Participants in the end-users sobriety and/or ongoing mental health support. The input may include phone numbers, social media handles, etc. In one embodiment, well established networks may include drop down menus for selection to national organizations, or local chapters in the individual's immediate geographic area. In one embodiment, past location data, social media posts, contacts, etc., may be analyzed for clues as to associations and established networks and such choices may be pre-filled or suggested by the app.

Once running (even if not registered or fully established as to baselines, networks or personal information) the app begins to gather data from various sources (e.g., opt-in for location tracking, step 884). For example, throughout the day, the system (app) may interact with the end-user. Such interaction may, for example, be based on the answers to the questions while setting the app up. In one embodiment, such interaction may include asking the same questions again or asking if the individual is feeling differently about any one or more of the questions. The app may, for example, ask the individual to report on their respective emotional health multiple times per day (e.g., step 886). The frequency may, for example, be determined by the answers during setup (e.g., an evaluation of the user's current level of recovery).

The emotional health may be captured multiple-ways, including, for example, through a personal device (smartphone), e.g., the user's mobile device on which the app is hosted (or via logon to a website, etc.). In one embodiment, the emotional query may take the form of a pop-up prompt, text message, or alert/notification with a response capability (e.g., radio button selections, link, etc.). For example, a user may be prompted to select one of multiple possible answers, such as requesting the user press one of any number (e.g., 7) of choices about their current emotional state (e.g., from Amazing to Poor . . . and one or more, even several options in between etc.). Such queries may occur multiple times per day (e.g., 7-15× per day), or at predefined junctures (morning, arriving at work, lunch, break time, etc.).

While capturing emotional state remains an important priority of various embodiments of the app, the frequency of direct user-app interaction in capturing emotional state may be varied depending on the stage of the user's recovery. For example, a sliding scale of a mix of direct questioning/queries and automated data collection transitioning to more automated collection as the user progresses in recovery. Such a transition may be further facilitated as the app learns or becomes more familiar with the user's habits and activities compared to established emotion states in similar circumstances.

Such queries may include actual actions over answering question, such as requests to send a selfie or face shot. Such photos may be logged and entered into facial or emotional recognition system supported by, for example AI for analysis of the individual's emotional state. The photos may be correlated to past events and past photos to recognize facial trends leading up to highly charged emotional states and/or potential relapse or other events. In one embodiment, social media images are analyzed for similar purposes, and the system may also commandeer the user's mobile device camera (with privacy permissions appropriately set, for example) to evaluate facial expressions (e.g., FEA), features, etc., at other times as well.

In other embodiments, other interactions of various calibers may be utilized or recognized, and such other interactions are not necessarily direct. For example, the app may collect information from other apps from which it has appropriate permission. Such collected information may be, for example, a user's record of wins/losses in an online game, or an amount of playing time, for example. It may be inferred upon experience or other factors what the significance of any such interaction may be with respect to the user's emotional state/health, but presumably, successful completion of certain games may be viewed positively and provide some data for a complete picture of emotional health and leaving a game incomplete (particularly if a history shows completion combined with good emotional status). The same data may be combined with other events, which if negative, (particularly if the game playing is seen as negative, as in on-line addiction, for example) may point to a less stable or more difficult emotional health situation.

Accordingly, the app may collect data by direct inquiry, background activities (locations, purchases, etc.), and through, for example, use of other apps or facilities. In one embodiment, direct inquiry may be the primary (or single) source of emotional data. In other embodiments, a variety of sources are tied together to produce an emotional score on which other decisions (e.g., assigning action items) may be based or modulated. The various embodiments may include or be described, for example, as collecting data in the background and preparing information supporting overall structure/habits of the individual, and such structure/habits may provide a baseline from which to compare new events affecting the individual's emotional status.

For example, the app may monitor the end-user/individual's typical days from the time they get up, to what they do daily and where they do it. Such activities/locations may be reported/recorded in a comprehensive manner. Consistency within such activities/locations may be rewarded. The app may, for example, keep a running score of points or a percentage value reflecting how well the individual is able to stick with a consistent program. Such score may be reported to the individual through pop-ups or via a main page of the app. The current score may be shown on an icon representing the app on the user's mobile device home page. A good score or high percentage indicates the user has been staying on track, has consistency, and/or is essentially running a good program/life and/or exhibiting, for example, good personal accountability. Such scores may be reported, for example, to stakeholders, insurance companies, etc. Progress or good scores may be re-numerated with gifts, coupons (e.g., “Your local Starbucks says congratulations—coffee on us!”), or cash (even small amounts) from stakeholders, insurance companies, or community organizations for example.

Rewards in the form of virtual badges, trophy's or other status may be supplemented with a physical letter or shout out at a local meeting (e.g., meeting leader notified via text/notification for example). Charts may be developed and shared with a user to show progress over a week, month, or other time frame—further reinforcing progress/status. The app may include facilities to receive appeals or requests for further points based on circumstances or specific challenges which the individual has successfully navigated.

Inconsistency of normal habits or routines or other indications may point to a relapse event. Other indications may potentially include an outright admission by the user (e.g., “I need a drink”) which may be an answer the user gives to one of the periodic questions, or a statement captured on an open mic. In such cases, the user's network is notified, or the app takes other action to help move the user past the current situation (e.g., telling the user to change his location immediately, or check-in to a specific sobriety group, etc.).

Data collected may be correlated to other data and to deeper patterns that, while may be applicable to the broader class of individuals or even applicable to all users, but most applicable to the individual user may be identified by the app and shared with the individual, his/her counselors, medical professionals, etc. Such deeper patterns may be, for example, a realization (or identification by the app) that going out to certain activities (e.g., staying up too late) throw off an individual and puts them in relapse danger. Such identification of areas where emotional deviations occur will identify much more detail about the real problem(s) the individual is facing (and potentially other user's as well)—thus reaching to the core, deeper issues that root many Drug and Alcohol problems (i.e., the drug/alcohol problem being the symptom). Accordingly, the app (and an overall premise that may be applied) collects habits, movements, and other data about the user and activities, events, etc., along with their personal emotional state, including that emotion state as the user feels it/as they understand it/as they respond to it. The identifier patterns, triggers or other information is then applied to decisions about future events, notifications of stakeholders, response teams, etc.

In operation, various features or embodiments will play-out depending on activities and circumstances as they develop relative to the individual. For example, tracking movements of an individual and identifying potential issues with an establishment or part of an establishment visited. An evaluation may be performed to determine a level of response, which may be, for example, a text or alert asking the individual are you on-track for this evenings goals—any issues please call,” for example. As the day progresses a more serious event r circumstance may arise which may elevate the response to a direct call, or the app taking control of the individuals mobile device to start streaming audio and/or video to a help center who may then quickly decide if the situation is elevate to a full intervention or other means to alter the individual's current course. Such events may include, for example, detection of suspicious patterns around a bar or known drug source neighborhood and feedback from the individual's mobile device (or individual's automobile's on board system) indicating driving.

The application or other software may be running on the mobile device or hosted remotely (e.g., accessed via an API, subscription, or pay service call). National statistics, alcohol and chemical dependency best practices, geospatial technology and the ability to capture individual end-user data may be built into the application or accessed via local or remote storage and such data sets may be utilized in comparing the individual's data and/or guide decisions on a level of intervention or support to be issued.

For example, these data sets, coupled with a collection of real-time emotional cues, may be used to arm recovering individuals with a personalized app experience. Thus, the app may identify specific problematic relapse triggers based on individual data by detecting potential relapse cues and triggers prior to them becoming a full relapse episode. The same may be compared or evaluated against national statistics and the individuals known prior history.

In various embodiments, the application is customizable by the end-user and can be accessed by any personal device. Such access may be over the Internet or be an installed Android/iOS application. By collecting and processing a myriad of emotional, psychological, geographical, and social matrix, end users are in a unique position to monitor, track, and react to emotional deviations that can lead to relapse. Again, such collection may include seemingly unrelated activities and can be correlated back to events and used for projections under similar or other circumstances.

In addition, the app may be configured to alert multiple participants who are stakeholders in the end-users recovery process. Stakeholders are participants in the end-users “recovery network”. Because the app is highly customizable, various participants within the end-users recovery network can be notified based on a number of criteria, including but not limited to, the severity of relapse danger, relapse location, and their relationship to the end-user. The app is a companion for recovery support, fits perfectly (fits well, for example), and can aid in alcohol and drug-related support groups. The app may include capabilities in support of the above, which may include, for example and one or more of:
Take control of phone to send images to counselor
Take control of phone to get immediate questions answered (therapist calls, Mr. X, you stopped near a liquor store, let's talk.”)
Real-time credit card information. Purchases.
Link to liquor drug sales outlets
GPS coordinated map of all liquor/tobacco/cannabis/etc. sales. Areas of town known for drug sales. Locations of beer in grocery stores.
Geofencing—notifications, alerts, not allowed to pass, etc.
Take over phone to get images/audio of activities.
Signed waiver of privacy—user selects acceptable level.
Warn user before entering a liquor zone. Call from app, call from autonomous voice, call from stakeholder, Group call—e.g., stakeholder, therapist, AAA co-member. May be based, for example, on severity of event.
Differentiate from pre-cursor and full-on trigger events.
Different customized response, from autonomous virtual assistant to full on intervention.
Full on intervention with QRF—rapid response team on the way to location.
Full on virtual intervention group chat call when needed.
Provision of automated reports to providers, stakeholders, individuals/patients, insurance companies, etc.
Billing to insurance companies based on performance (e.g., collecting bonuses for miles stones, days sober, etc.).
Managing groups of intervenors, paid response forces for high end implementations and/or volunteers, and volunteer forces trained or guided for community based service organizations, for example.
Dispatching an intervention APB (All Points Bulletin) that may include, for example, last known location, pictures, bio, known circumstances, and predicted current issue. An APB alerts responding forces as to potential issues, background etc. and. For example a suggested intervention style. The application may organize the intervention along proper guidelines (e.g., making sure two people are present and available and confirming, tracking their locations, turning all cell phone cameras on, for example—many variations on the protocols may be implemented.)

The significant amounts of data collected (and utilized) by the app and may be used for reports or other paperwork relevant to a user's rehab, insurance, etc. Such data is extensive and is able to provide insight in to the user's overall well-being and may be analyzed or reviewed to find patterns or underlying issues which may be the root cause of the dependency issues to begin with. Such analysis may be performed by an analysis module that searches for activity/emotional patterns related to one or more relapse or low emotional state events in the user's data history and the results may be provided to counselors or other authorized health professionals (or directly to the user) depending on permission levels set in the user's preferences, for example.

Further, the application and its various embodiments may be applied in a general health and well-being use for users not associated with or having dependency issues. The user may utilize the app to help realize various emotional cycles and provide a toolkit to help deal with the ups and downs of daily life and activities which can be especially overwhelming in the modern world.

Although the various embodiments have been described herein with reference mainly to dependencies such as alcohol and/or drug abuse any type of dependency may be addressed by the same and/or similar processes.

In describing the embodiments, and as illustrated in the drawings, specific terminology is employed for the sake of clarity. However, the various embodiments are not intended to be limited to the specific terminology so selected (or drawings provided), and it should be understood that the ordinarily skilled artisan may utilize similar, related, or even different terminology depending on the embodiment or selected topic therein to discuss, describe, or implement the same. Further, it should be understood that each specific element includes all technical equivalents which operate in a similar manner, as will be understood by the artisan. For example, when describing a mobile device (e.g., cell phone), any other equivalent device, such as a notebook, vehicle communication system (e.g., on-star), tablet, smart TV, or other device having an equivalent function or capability, whether or not listed herein, may be substituted therewith. Furthermore, the system and processes may be conveniently spread across a plurality of devices including various sensors that collect data used by the system and/o app. Still further, the inventor recognizes that newly developed technologies not now known may also be substituted for the described parts and still not depart from the scope of the present invention. All other described items, including, but not limited to processes, data collection, communications, protocols, responses, etc. should also be considered in light of any and all available equivalents.

Portions of the various embodiments may be conveniently implemented using a conventional general purpose or a specialized digital computer or microprocessor programmed according to the teachings of the present disclosure, as will be apparent to those skilled in the computer art.

Appropriate software coding can readily be prepared by skilled programmers based on the teachings of the present disclosure, as will be apparent to those skilled in the software art. The various embodiments or portions thereof, may also be implemented by the preparation of application specific integrated circuits or by interconnecting an appropriate network of conventional component circuits, as will be readily apparent to those skilled in the art based on the present disclosure. Such circuits may include, for example, biometric sensors (e.g., wearables, patches, implants, touch sensitive devices, etc.), chemical detectors, reflex tests/observations, IR scanners (e.g., Flir camera images), etc. Any or all of which may be combined with data points or other information as described above to analyze current situations or provide information for later analysis and/or comparisons.

The various embodiments include a computer program product which is a storage medium (media) having instructions stored thereon/in which can be used to control, or cause, a computer to perform any of the processes of the embodiments. The storage medium can include, but is not limited to, any type of disk including floppy disks, mini disks (MD's), optical discs, DVD, HD-DVD, Blue-ray, CD-ROMS, CD or DVD RW+/−, micro-drive, cloud storage, and magneto-optical disks, ROMs, RAMs, EPROMs, EEPROMs, DRAMs, VRAMs, flash memory devices (including flash cards, memory sticks), magnetic or optical cards, SIM cards, MEMS, nanosystems (including molecular memory ICs), RAID devices, remote data storage/archive/warehousing, or any type of media or device suitable for storing instructions and/or data.

Stored on any one of the computer readable medium (media), the embodiments may include software for controlling both the hardware of the general purpose/specialized computer or microprocessor, and for enabling the computer or microprocessor to interact with a human user or other mechanism utilizing the results of any embodiment or variations/equivalents thereof. Such software may include, but is not limited to, device drivers, operating systems, and user applications. Ultimately, such computer readable media further includes software for performing any embodiment as described above and equivalents thereof.

Included in the programming (software) of the general/specialized computer or microprocessor are software modules for implementing the teachings of the various embodiments, including, but not limited to, identification of environment and other circumstances, retrieval of stored information and comparison to statistical data and past circumstances in support of any of the above, and the display, storage, or communication of results according to the processes as described herein and equivalent processes whether or not described herein.

The embodiments may suitably comprise, consist of, or consist essentially of, any of element (the various parts or features of the embodiments and their equivalents as described herein). Further, the embodiments illustratively disclosed herein may be practiced in the absence of any element, whether or not specifically disclosed herein. Obviously, numerous modifications and variations of each embodiment are possible in light of the above teachings. It is therefore to be understood that within the scope of any claims, the invention, or any embodiment thereof, may be practiced otherwise than as specifically described herein.

What is claimed is:

1. An application configured to collect data related to any of physiological, emotional, and environmental circumstances of the individual, compare the data to statistically relevant pool of knowledge derived from any of other individuals, other like individuals, and past data of the individual to identify a probability of a relapse and a relative danger/impact of the potential relapse to decide an appropriate course of action which may include any of an automated virtual assistant response, call from a healthcare professional, or a full intervention.

2. The application according to claim 1, wherein the application includes preferences that may be adjusted by the individual and/or required by a program in which the individual is enrolled.

3. The application according to claim 1, wherein the application is in communication with biometric data gathering apparatus linked to the individual.

4. The application according to claim 1, wherein the application or a remote process manages an intervention by contacting and verifying responders including a mix of professionals, volunteers, and stakeholders.

5. The application according to claim 1, wherein the data collection comprises physiological data from a wearable device.

6. The application according to claim 3, wherein the biometric data gathering apparatus comprises a Galvanic Skin Response (GSR) detection apparatus.

7. The application according to claim 3, further comprising an AI module configured to correlate GSR data to events and/or emotion cues to predict a current status of the user and invoke at least one of a query and interventional response if such prediction indicates a relapse event may occur or has occurred.

8. A system of monitoring emotional health comprising a smartphone configured to collect physiological and emotional data about a user, utilize the collected data to determine a user's likelihood of a relapse, and coordinate a response before, during, or shortly after the relapse.

9. The system according to claim 8, wherein the physiological data is derived from a fitness-like device comprising one of a Fitbit, heart rate monitor, blood pressure device, galvanic skin response detector, treadmill, stairmaster, stationary bike, an electronic system at a gym, or like devices/systems.

10. The system according to claim 8, further comprising an emotional query module configured to ask a user how they are feeling.

11. The system according to claim 10, wherein the user is queried at one of regular time slots, periodic time slots, random time slots, upon events which may be associated with changes in emotional state, and events upon which the user has a history of emotional state changes, and wherein query responses combined with empirical data collected about the user's condition are together compared to previous similar conditions and emotional states to determine the relapse likelihood.

12. The system according to claim 11, wherein said events are combinations of more than one event or factor that affect emotional status.

13. The system according to claim 8, further comprising a notification module configured to alert at least one of medical personnel, stakeholders, or family member if a relapse appears likely.

14. The system according to claim 13, wherein the alert comprises information about the user's current condition and location data of the user.

15. The system according to claim 8, further comprising an emotional cue module configured to query the user/individual for emotional status information including a snapshot or video comprising at least one of a selfie, facial shot, circumstances, or surroundings.

16. The application or system according to claim 15, wherein the emotional cue module records interaction of the user with the emotional cue module, and, in the event of a deviation or other concerning circumstances, forwards the recorded interaction to a health professional.

17. The application or system according to claim 15, wherein the emotional cue module records user/individual interaction with the app or system via a mobile device camera along with contemporaneous biometric data.

18. The application or system according to claim 17, further comprising an analysis of the photos or videos to establish or confirm an emotional state of the user/individual, such analysis including consideration of the contemporaneous biometric data including galvanic skin response.
